(19) United States
(12) Patent Application Publication
(10) Pub. No.: US 2026/0245677 A1
(43) Pub. Date: Aug. 20, 2026

(54) SYSTEMS AND METHODS FOR MACHINE LEARNING BASED ALLOY DEVELOPMENT

(71) Applicant: Phaseshift Technologies Inc., Toronto (CA)

(72) Inventors: Gurjot Singh Dhaliwal, Toronto (CA); Abu Anand, Toronto (CA); Zhen Li, Hamilton (CA); Osazee Ewaen Ero, Waterloo (CA)

(21) Appl. No.: 19/545,366

(22) Filed: Feb. 20, 2026

Related U.S. Application Data
(60) Provisional application No. 63/760,898, filed on Feb. 20, 2025.

(51) Int. Cl. G16C 60/00; G06F 30/27; G06F 111/06; G16C 20/30; G16C 20/70

(52) U.S. Cl. CPC G16C 60/00; G06F 30/27; G16C 20/30; G16C 20/70; G06F 2111/06

(57) ABSTRACT

Methods and systems for developing one or more lead alloy compositions based on a set of design objectives comprising receiving alloy composition data for one or more input alloys; identifying a set of the alloy composition features; determining the material behavior of each input alloy based on the set of alloy composition features; determining a plurality of candidate alloy compositions having material behavior conforming to a first subset of the design objectives; applying a multiscale simulation process to evaluate each candidate alloy composition; identifying, a first set of one or more simulated alloy compositions that satisfy the first subset of the design objectives; identifying, a second set of one or more simulated alloy compositions from the first set based on a second subset of design objectives; and validating each of the second set through experimental evaluation and determining the one or more lead alloy compositions.

This application claims the benefit of U.S. Provisional Patent Application No. 63/760,898 filed on Feb. 20, 2025, entitled “Systems and Methods for A Rapid Alloy Development Platform”. The entirety of U.S. Provisional Patent Application No. 63/760,898 is incorporated herein by reference.

### FIELD

The present disclosure generally relates to an alloy development process, and in particular, to methods and systems for enabling an alloy development process using machine learning and multiscale simulation models.

### INTRODUCTION

The following is not an admission that anything discussed below is part of the prior art or part of the common general knowledge of a person skilled in the art.

Alloys combine two or more elements, typically metals, to create a material that has superior or more desirable characteristics than its individual components. This makes alloys versatile and valuable in a wide range of applications. For example, in the field of manufacturing, cemented carbides (or cermets) consisting of a hard ceramic particle embedded in binder alloy have been used for its exceptional hardness, wear resistance, and high-temperature stability. This makes the material ideal for demanding applications such as cutting tools and wear-resistant components. The metal binder adds toughness and ductility, preventing the otherwise brittle carbides from fracturing under impact or stress by holding the particles together and absorbing shock. This combination results in a material with both hardness and toughness, capable of performing under extreme conditions.

With the advancement of manufacturing techniques, traditional alloys such as bronze, brass, and some cermet alloys, for example, have become insufficient for supporting emerging applications. Modern alloy compositions with unique material properties are necessary for performing specific tasks that align with the narrow design requirements. However, designing new alloy compositions can be costly and time-consuming both theoretically and practically. Among other restrictions, it can be burdensome to physically create an alloy and verify its material properties to ensure high performance and compliance.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In one broad aspect, in accordance with some embodiments, there is generally provided a method for developing one or more lead alloy compositions based on a set of design objectives defining a target alloy performance. The method comprises receiving, at a processor coupled to a memory, alloy composition data for one or more input alloys, the alloy composition data corresponding to a material behavior of each alloy and the one or more input alloys comprising material properties associated with a first subset of the design objectives. The method further comprises identifying, at the processor, from the received alloy composition data from the one or more input alloys a set of the alloy composition features related to the target alloy performance. The method further comprises applying, at the processor, a prediction data model to determine the material behavior of each input alloy based on the set of alloy composition features. The method further comprises applying, at the processor, an optimization data model to determine a plurality of candidate alloy compositions having material behavior conforming to the first subset of the design objectives. The method further comprises applying, at the processor, a multiscale simulation process, to evaluate the material properties and material behavior of each candidate alloy composition, the multiscale simulation process comprising at least two simulation models operating in a pre-determined sequence, wherein an input of a second simulation model is based on an output of a first simulation model in the pre-determined sequence. The method further comprises identifying, at the processor, a first set of one or more simulated alloy compositions that satisfy the first subset of the design objectives. The method further comprises identifying, at the processor, a second set of one or more simulated alloy compositions from the first set based on a second subset of the design objectives. The method further comprises validating, at the processor, the material properties and the material behavior of each of the second set of one or more simulated alloy compositions through experimental evaluation and determining the one or more lead alloy compositions based on the experimental evaluation.

In some embodiments, the alloy composition data comprises simulated alloy composition data generated from the multiscale simulation process.

In some embodiments, applying, at the processor, the optimization data model further comprises identifying a set of alloy compositions that do not conform to the first subset of the design objectives and inputting simulated alloy composition data to the data optimization model and refining the optimization data model based on the simulated alloy composition data.

In some embodiments, the optimization data model is configured to assess at least two of the material properties in the first subset of the design objectives.

In some embodiments, applying the multiscale simulation process, at the processor, further comprises inputting generative machine learning-based structures based on the simulated alloy composition data.

In some embodiments, applying the multiscale simulation process, at the processor, further comprises sequencing the simulation models based on a material scale.

In some embodiments, the at least two simulation models further comprise at least four models applied in a pre-determined sequence in increasing scale from an atomic scale to a macro scale.

In some embodiments, at least one of the at least two simulation models is based on machine learning methods.

In some embodiments, applying the multiscale simulation process, at the processor, further comprises selecting the simulation model for the pre-determined sequence based on a scale of atoms.

In some embodiments, an input of a third simulation model is based on the output from the first simulation model.

In some embodiments, the simulated alloy composition data generated from the multiscale simulation process is stored in the memory.

In some embodiments, the second subset of the design objectives corresponds to material behaviors associated with a feasibility of experimental evaluation.

In another broad aspect, in accordance with some embodiments, there is generally provided a system for developing one or more lead alloy compositions based on a set of design objectives defining a target alloy performance. The system comprises a prediction data model, an optimization data model, at least two simulation models, and a processor coupled to a memory. The processor is configured to receive alloy composition data for one or more input alloys, the alloy composition data corresponding to a material behavior of each alloy and the one or more input alloys comprising material properties associated with a first subset of the design objectives; identify from the received alloy composition data from the one or more input alloys a set of the alloy composition features related to the target alloy performance; apply the prediction data model to determine the material behavior of each input alloy based on the set of alloy composition features; apply an optimization data model to determine a plurality of candidate alloy compositions having material behavior conforming to the first subset of the design objectives; apply a multiscale simulation process to evaluate the material properties and material behavior of each candidate alloy composition, the multiscale simulation process comprising at least two simulation models operating in a pre-determined sequence, wherein an input of a second simulation model is based on an output of a first simulation model in the pre-determined sequence; identify a first set of one or more simulated alloy compositions that satisfy the first subset of the design objectives; identify a second set of one or more simulated alloy compositions from the first set based on a second subset of the design objectives; and receive experimental validation data comprising material behavior of each of the second set of one or more simulated alloy compositions and provide recommendations for determining one or more lead alloy compositions based on the experimental validation data.

In some embodiments, the alloy composition data comprises simulated alloy composition data generated from the multiscale simulation process.

In some embodiments, the processor is further configured to apply the optimization data model to identify a set of alloy compositions that do not conform to the first subset of the design objectives; input simulated alloy composition data to the data optimization model; and refine the optimization data model based on the simulated alloy composition data.

In some embodiments, the processor is further configured to apply the optimization data model to assess at least two of the material properties in the first subset of the design objectives.

In some embodiments, the processor is further configured to apply the multiscale simulation process to input generative machine learning-based structures based on the simulated alloy composition data.

In some embodiments, the processor is further configured to apply the multiscale simulation process to sequence the simulation models based on a material scale.

In some embodiments, the at least two simulation models further comprise at least four simulation models applied in a pre-determined sequence from an atomic scale to a macro scale.

In some embodiments, at least one of the at least two simulation models is based on machine learning methods.

In some embodiments, the processor is further configured to apply the multiscale simulation process to select the simulation models for the pre-determined sequence based on accuracy and speed.

In some embodiments, the processor is further configured to apply the multiscale simulation process to select the simulation models for the pre-determined sequence based on a scale of atoms.

In some embodiments, the input of a third simulation model is based on the output from the first simulation model.

In some embodiments, the simulated alloy composition data generated from the multiscale simulation process is stored in the memory.

In some embodiments, the second subset of design objectives corresponds to material behaviors associated with a feasibility of experimental evaluation.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1A shows a schematic diagram of an example alloy development process in accordance with some embodiments.

FIG. 1B shows a schematic diagram of an example alloy development system in accordance with some embodiments.

FIG. 2 shows an example server in accordance with some embodiments.

FIG. 3A shows a flow diagram of an example method for alloy development in accordance with some embodiments.

FIG. 3B shows another flow diagram of an example method for alloy development in accordance with some embodiments.

FIG. 4 shows a flow diagram of an alloy development process in accordance with some embodiments.

FIG. 5 shows an example flow diagram of a multiscale simulation process in accordance with some embodiments.

FIG. 6 shows a table of example design requirements for a target alloy in accordance with some embodiments.

FIG. 7 shows a table of application areas for an alloy development process.

FIG. 8 shows example phase diagrams for an example alloy composition in accordance with some embodiments.

### DESCRIPTION OF VARIOUS EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

It should also be noted that the terms “coupled” or “coupling” as used herein can have several different meanings depending in the context in which these terms are used. For example, the terms coupled or coupling can have a logical, mechanical, fluidic or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore, coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Exemplary communicative couplings include, but are not limited to, logical couplings, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

The example systems and methods described herein may be implemented as a combination of hardware or software. In some cases, the examples described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

Some elements that are used to implement at least part of the systems, methods, and devices described herein may be implemented via software that is written in a high-level procedural language such as object-oriented programming. The program code may be written in C⁺⁺, C#, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having at least one processor, an operating system, and the associated hardware and software that is used to implement the functionality of at least one of the methods described herein. The software program code, when read by the device, configures the device to operate in a new, specific, and predefined manner (e.g., as a specific-purpose computer) in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

As industries continue to evolve and diversify, the demand for innovative alloys tailored to specific use cases and applications has grown exponentially. These advanced materials are critical for sectors such as aerospace, automotive, energy, and healthcare, among others, where performance, durability, and sustainability are paramount. However, the discovery of new alloys often presents a significant challenge. For example, each new material requires extensive experimentation to evaluate its properties, such as strength, corrosion resistance, and thermal stability, etc., under various conditions. This trial-and-error approach is both time-consuming and resource-intensive, often slowing down the pace of innovation. To meet the increasing industrial needs, there is a pressing demand for more efficient methods to identify new alloy compositions, predict alloy behaviors and streamline the development process.

Further, new manufacturing techniques can require specific alloys to support emerging techniques. For example, laser powder bed fusion (LPBF) is a type of metal additive manufacturing process that uses a high-powered laser to selectively melt and fuse metal powder layer by layer to create complex, high-precision parts. This technique allows for production of intricate geometries that are difficult or impossible to achieve with traditional manufacturing methods. The process offers high design flexibility and material efficiency, with applications in industries such as, but not limited to, aerospace, automotive, and medical. LPBF produces parts with excellent mechanical properties, through challenges including managing stresses, controlling microstructure, and defects in the printed parts. However, certain traditional alloys present several challenges for the LPBF

process. For example, traditional cemented carbides, such as a metal matrix composite consisting of a tungsten carbide ceramic particle embedded in a cobalt metal binder presents at least the following challenges for the LPBF process: 1) tungsten carbide and other carbides have significantly higher melting points than the metal binder making it difficult to achieve uniform melting, often leading to defects; 2) cemented carbides are prone to thermal cracking due to the mismatch in thermal expansion between the hard carbide particles and the more ductile metal binder; accordingly, during LPBF, the rapid heating and cooling cycles can induce high residual stresses, leading to cracking or distortion in the printed part; and 3) maintaining a high density in printed parts is crucial to structural integrity and optimal mechanical performance. However, LPBF often produces parts with internal porosity, especially in composite materials like cemented carbides. This can occur when materials primarily designed for another process are used with LPBF.

Accordingly, it is desirable to develop an alloy optimized for use in emerging additive manufacturing techniques, such as the LPBF process. However, designing alloys to meet a specific target alloy performance specification is challenging, costly, and time consuming.

Furthermore, it can be difficult to predict the mechanical properties and stability of any given alloy. The alloy composition may exhibit different material properties than the two metallic elements it comprises. Further, an alloy can have different material properties and consequently different material behaviors, depending on the weight percentage blend of its constituent metallic elements. Thus, even though the material properties of a particular metallic component may be known, it may not be clear how one metallic component will interact with a different metallic component when joined together in an alloy composition.

To understand alloy material properties, alloy designers can look to experimental data available in the literature. However, this data may include errors, information gaps, and contain irrelevant information that may not be related to the design requirements for a specific project. In addition, information for specific alloys may be very limited in published literature. Some metallic elements can be difficult to procure due to supply, prohibitively expensive to obtain in large quantities, or practically difficult to work with (e.g., volatile, too heavy, needs specific conditions to be workable). Even with the data available from the literature, there may not be enough information to develop and experimentally validate an alloy suited for a specific purpose (e.g., emerging additive manufacturing techniques such as LPBF). Such alloys may not be well documented as there was no previous need for an alloy with those properties.

Further, it may be risky to actualize certain alloy compositions in the real world due to supply and cost constraints. Theoretical material properties can only provide limited insights or information; certain material properties can only be determined after the alloy has been created. Although there are techniques for simulating the properties of alloys, these techniques are often limited to simulating a single scale of the alloy (i.e., the nano scale or the micro scale). Such techniques often also do not account for the relationships between material scales that help determine the real-world properties of a given alloy.

Also, although simulation can be a useful tool for developing candidate alloys, the simulation process is computationally intensive and can take days (or longer) to accurately simulate a given alloy. In addition, it can be challenging to determine which alloy compositions are useful for simulating. Without an intuition for the underlying physics for a given alloy, computational resources could be unnecessarily wasted.

The various embodiments disclosed herein describe machine learning-based alloy development systems and methods capable of determining alloy compositions based on one or more design objectives, thereby streamlining the process of determining an alloy suited for a particular application. The alloy development systems and methods disclosed herein can, in some embodiments, use one or more data models to identify and extract material properties from a database of known alloy compositions or from simulated alloy compositions. The presently disclosed systems and methods can use one or more data models to determine candidate alloy compositions having material behavior conforming to a set of design objectives.

Furthermore, in the various embodiments disclosed herein, the alloy development systems and methods can apply a multiscale simulation process to verify and validate the material properties of an alloy at various material scales. In particular, the multiscale simulation process in the presently disclosed systems and methods can also use various simulation models in a pre-determined sequence to determine the material behavior of an alloy across different material scales. The presently disclosed systems and methods can identify and validate one or more sets of simulated alloy compositions that satisfy a set of design objectives. The presently disclosed systems and methods can determine one or more lead alloy compositions based on experimental validation.

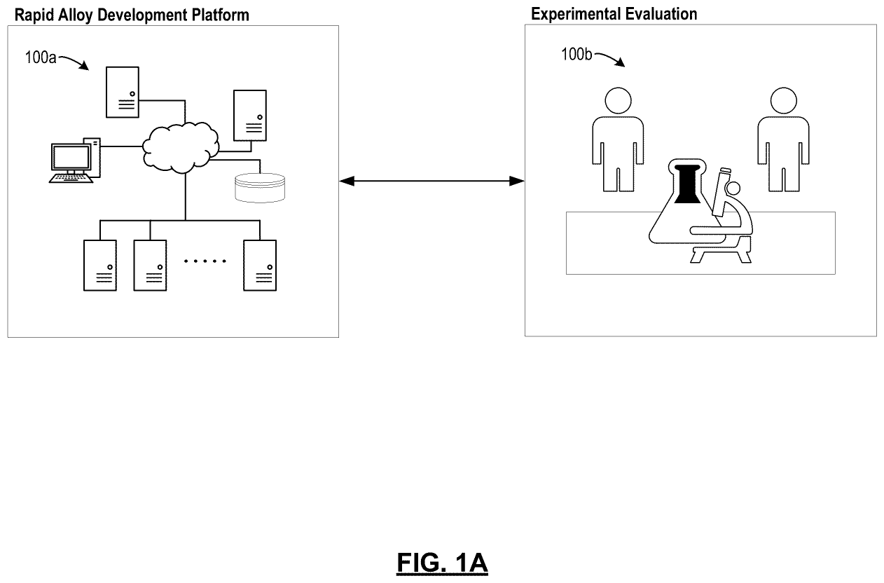

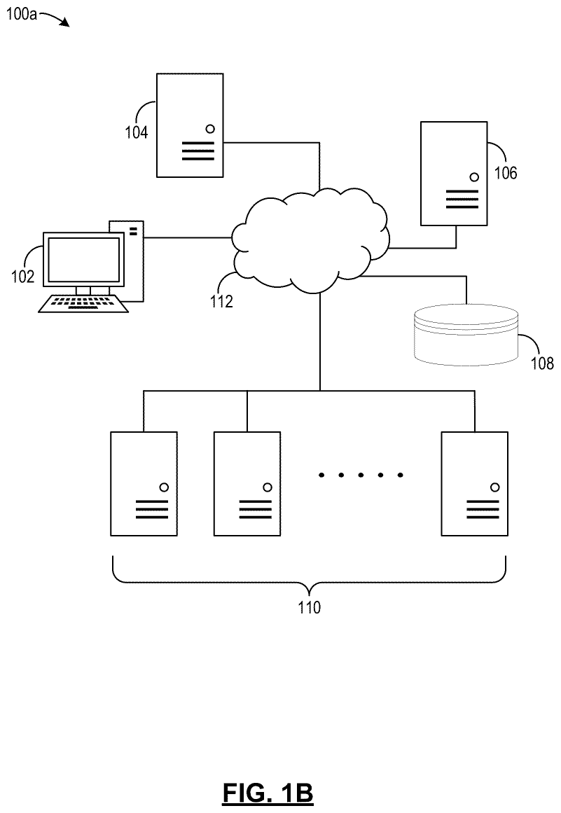

Reference is first made to FIG. 1A, which provides a general overview of the application of an alloy development process performed by system 100a. In accordance with some embodiments of the disclosed invention, the system 100a performs an alloy development process that identifies one or more lead alloy compositions based on a set of design objectives defining one or more target performance criteria. The one or more lead alloy compositions can be validated using experimental evaluation 100b to verify the material properties and corresponding material behaviors of the one or more lead alloy compositions. In some embodiments, system 100a can receive experimental evaluation data from the experimental evaluation 100b. System 100a can then provide recommendations for determining one or more lead alloy compositions based on the received experimental evaluation data. For example, alloy composition data determined from the experimental evaluation 100b can be incorporated into the system 100a to refine or improve the alloy development process.

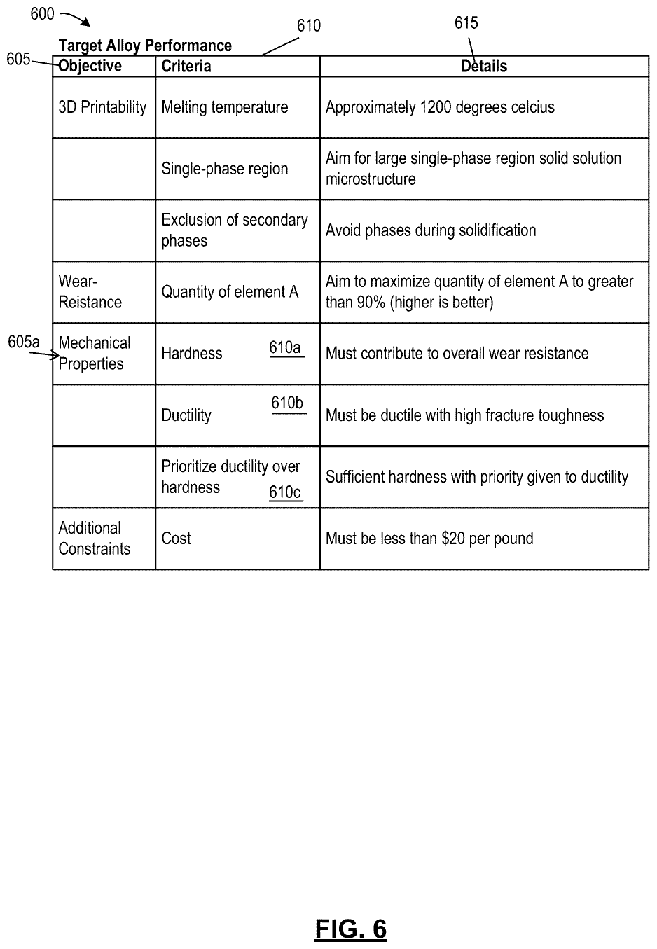

System 100a can develop one or more lead alloy compositions based on a set of design objectives. The design objectives specify a target alloy performance required for a particular application. In general, the target alloy performance is established by correlating the design objectives with one or more performance criteria. The target alloy performance may also account for additional constraints, such as, cost limitations, material availability and accessibility, manufacturing process requirements, etc.). FIG. 6 shows a table of example design objectives for a target alloy in accordance with some embodiments.

Design objectives are listed in the first column 605 of the table shown in FIG. 6. The objectives define broad features that are desirable for use in a field of application. For example, developing an alloy composition for LPBF may have four main objectives: 1) usability in three-dimensional printing applications; 2) high wear resistance; 3) specific mechanical properties; and 4) additional constraints. It will be understood that there may be more or fewer design objectives required depending on the target application of the alloy. Design criteria are listed in the second column 610 of the table shown in FIG. 6. Design criteria define material properties or material behavior that need to be exhibited by the one or more lead alloy compositions to satisfy the design objectives. For example, to satisfy the mechanical properties objective 605a listed in the first column 605, the alloy composition may need to be of a certain hardness 610a, have a certain ductility 610b, and have optimal binding characteristics with certain metallic binders. In some cases, material properties may have inversely proportional relationships. For example, generally increasing the hardness of a material will decrease the ductility of the material. In some embodiments, the system 100a is configured to assess at least two material properties in the set of design objectives. As described above, in some cases, the design criteria may need to optimize between two conflicting material properties (e.g., hardness and ductility). In such cases, the design criteria can define which material property to prioritize 610c. It will be understood that there may be more or less design criteria depending on the target application of the alloy and the nature of the related design objectives.

Additional details are listed in the third column 615 of the table shown in FIG. 6. Additional details can further define the requirements of the design objectives and design criteria. For example, to maximize the wear-resistance of an alloy, the alloy may need a certain quantity of a metallic element present in the one or more lead alloy. The ‘details’ column may provide, for example, specific quantitative and/or qualitative values required to satisfy the design criteria and design objective. In other cases, the ‘details’ column may provide an upper threshold or a lower threshold, or a preferred range of values.

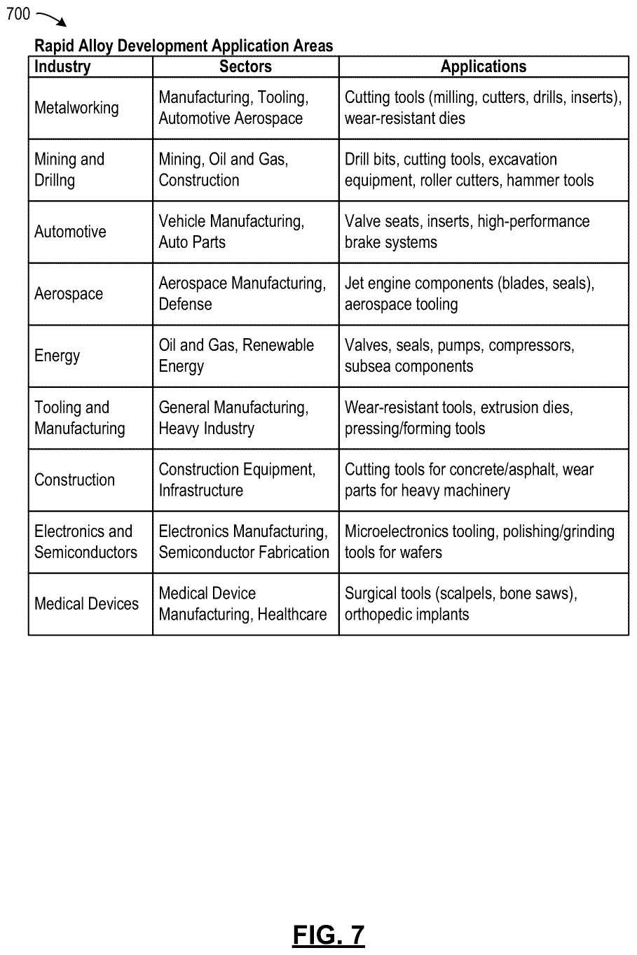

FIG. 7 shows a non-exhaustive list of industries in which the alloy development process disclosed in the various embodiments can be applied. In addition, the sector and specific application are also shown by way of example. The target alloy performance, and consequently, the design objectives can vary depending on the application. For example, alloys used in the automotive industry often have distinct performance objectives compared to those used in the medical devices industry. Additionally, even within the same industry, different companies or alloy manufacturers may define varying performance objectives for their alloy products, tailored to specific applications and market segments. These products may also be offered at different price points to meet diverse customer needs.

Reference is next made to FIG. 1B, which shows a schematic diagram of an example alloy development system in accordance with some embodiments. System 100a includes a server 102, a prediction data model 104, an optimization data model 106, a database 108, and one or more simulation models 110. Server 102, the prediction data model 104, the optimization data model 106, the database 108, and the one or more simulation models 110 can be connected to each other through network 112.

The prediction data model 104 can be configured to determine material behavior of one or more input alloys as described herein. Material behavior can be based on a set of alloy composition features related to the target alloy performance.

The optimization data model 106 can be configured to determine a plurality of candidate alloy compositions having material behavior conforming to a set of design objectives. The set of design objectives can correspond to desirable alloy properties for achieving the target alloy performance. Exemplary design objectives can include, but are not limited to, cost of components, cost of manufacturing, mechanical performance (e.g., hardness, ductility, conductivity, elasticity), and selection of manufacturing process. In some embodiments, design objectives can be clustered and/or combined as one or more subsets.

For example, a first subset of design objectives can correspond to material properties that align with target alloy performance, considering objectives such as mechanical performance. A second subset of design objectives can correspond to properties that cannot be validated and/or accounted for during simulation, considering objectives such as, but not limited to, feasibility of experimental validation, costs associated with manufacturing process, and selection of manufacturing process (e.g., angular particles versus spray dried).

The database 108 can be a memory for storing received alloy composition data. Received alloy composition data can be from experimentally available data (e.g., from public databases) or from simulated alloy composition data generated through a multiscale simulation process such as the multiscale simulation process described herein.

The one or more simulation models 110 can be configured to simulate the material properties and material behavior of an alloy composition at various material scales. For example, a first simulation model can be configured to simulate material properties and material behavior of an alloy composition at a nano scale, and a second simulation model can be configured to simulate material properties and material behavior at a macro scale.

The network 112 may be any network capable of carrying data, which may be connected via infrastructure such as Ethernet, RS-485, PROFIBUS, CAN Bus, USB, old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, and others, including any combination of these, capable of interfacing with, and enabling communication between any or all of the server 102, the prediction data model 104, and/or the optimization data model 106. Each of the server 102, prediction data model 104, and optimization data model 106 may be equipped with suitable network communication hardware so as to enable communications with each other through protocols such as MODBUS, IEEE 802.3, IEEE 802.11, DeviceNet, or any other communications protocol suitable for communication between industrial devices.

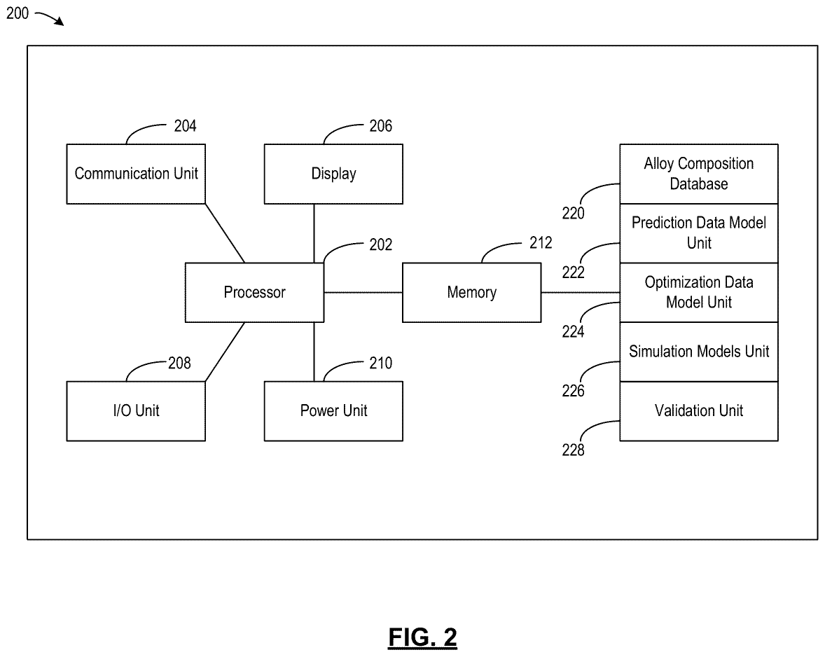

Reference is next made to FIG. 2, which shows a device diagram of an example server 102 of FIG. 1B in accordance with one or more embodiments. Server 102 can include a processor unit 202, a communication unit 204, an I/O unit 208, a power unit 210, and a memory 212.

The communication unit 204 can include any combination of hardware that enables wired or wireless connection capabilities. For example, the communication unit 204 can include a radio or network card that communicates using standards such as IEEE 802.3, IEEE 802.11, DeviceNet, MODBUS, and any other protocol suitable for communicating with industrial devices. The communication unit 204 can allow the server 102 to communicate with the prediction data model 104, the optimization data model 106, the database 108, the one or more simulation models 112, other devices, computers, local area networks, wide area networks, and external networks.

In the embodiment shown in FIG. 1B, server 102 includes hardware enabling communication through the network 112. Server 102 can then communicate to send and receive data, such as alloy composition data to the system, image data, alerts, text data, and more. For instance, server 102 may receive alloy composition data from one or more input alloys and relay the alloy composition data to the prediction data model 104, the optimization data model 106, the database 108, or the one or more simulation models 112.

The processor unit 202 controls the operation of the server 102. The processor unit 202 can be any suitable processor, controller or digital signal processor that can provide sufficient processing power depending on the configuration, purposes and requirements of the server 102 as is known by those skilled in the art. For example, the processor unit 202 may be a high-performance general processor, such as an Intel® processor or an AMD® processor. The processor unit 202 may preferably contain a graphics card or a dedicated graphics processing unit (GPU), such as an Nvidia® RTC 5000 Ada, for accelerating locally run machine learning models. The provision of a GPU may provide speed advantages for running machine learning algorithms such as material verification, object recognition, defect detection, bit verification, and more. In some embodiments, the processor unit 202 can include more than one processor with each processor being configured to perform different dedicated tasks.

The I/O unit 208 can include hardware for interfacing with at least one of a mouse, a keyboard, a touch screen, a thumbwheel, a trackpad, a trackball, a card-reader, an audio source, a microphone, voice recognition software and the like again depending on the particular implementation of the server 102. The I/O unit 208 may then facilitate taking user input to the server 102. In some cases, some of these components can be integrated with one another.

The power unit 210 can be any suitable power source that provides power to the server 102 such as a power adaptor or a rechargeable battery pack depending on the implementation of the server 102 as is known by those skilled in the art.

The memory 212 comprises software code for implementing, among other programs, alloy composition database 220, prediction data model unit 222, the optimization data model unit 224, the simulation models unit 226, and the validation unit 228. The memory 212 can include RAM, ROM, one or more hard drives, one or more flash drives or some other suitable data storage elements such as disk drives, etc.

The alloy composition database 220 can receive alloy composition data for one or more input alloys. An input alloy can be any alloy comprising a combination of base metals that can be used to form alloys such as, for example, iron, aluminum, copper, nickel, titanium, magnesium, and zinc. Other examples can include alloying elements such as chromium, carbon, silicon, manganese, molybdenum, vanadium, tungsten, cobalt, niobium, and zirconium. It will be appreciated that this is not an exhaustive list of possible metallic alloy components.

Each alloy can have alloy composition data corresponding to material behavior for the respective alloy. For example, material behavior can include behaviors such as, but not limited to, mechanical behavior (i.e., how a material responds to mechanical forces such as tension, compression and shear), thermal behavior (i.e., how a material responds to thermal forces such as increased temperature), electrical behaviors (i.e., how a material responds to electrical current), and chemical behaviors (i.e., how a material responds to environmental exposure).

Material behavior can be based on material properties. Material properties define the inherent attributes of the material. For example, material properties can include properties such as, but not limited to elasticity, plasticity, ductility, hardness, thermal conductivity, melting point, solidification point, resistivity, ferromagnetism, and corrosion resistance.

The system 100a can receive alloy composition data from various sources. For example, the prediction data model can receive alloy composition data from publicly available experimental data. In some cases, system 100a may not have enough information to determine the material behavior of each input alloy. Data related to certain alloys may be unstudied, inaccurate, or sparse in the published literature. In such cases, data can be generated to supplement insufficient alloy composition data using relevant simulations. For example, benchmark data generation can involve running simulations to generate data for a subset of known alloys with experimentally measured properties. This can help evaluate the accuracy of the simulations and identify any limitations in the modeling. This can also serve as a baseline to refine the simulation process.

As another example, initial data generation can be applied based on design requirements using Design of Experiments (DOE) methods. Techniques such as random sampling or quasi-random approaches can be used to explore a large search space while keeping computational costs manageable. This ensures that data is generated representative of the design landscape.

As another example, application-specific data can be generated which is data that is tailored to a particular application's requirements. The data is produced to align with the unique demands of the application.

Prediction data model unit 222 pre-processes received alloy composition data to be prepared for use with machine learning models such as the prediction data model 104. Pre-processing can include removing errors, gaps, or irrelevant information that does not apply to the particular application. Further, data from literature may be limited or provide inconclusive results. Such information can be filtered using techniques such as averaging.

Alloy composition data can also be featurized to transform it into a format that machine learning algorithms can process. This process captures the underlying physics of a material, which can be represented for example, through the physical properties of the alloy's elements and information about the bonds between elements and the bonds between elements and the material's crystal structure. Featurization uses mathematical functions to incorporate these elemental properties and structural information into columns. These organized columns allow machine learning algorithms to accurately predict material behaviors while embedding physical principles that improve a machine learning model's ability to understand the underlying science and generate new alloys.

Prediction data model unit 222 can apply machine learning methods to uncover complex relationships between alloy compositions and their material properties which can be difficult to identify using traditional methods. Machine learning provides a more reliable alternative by analyzing generated data and features to create mathematical models that can predict the properties of unknown alloys. Various machine learning algorithms are used to solve tasks such as predicting material properties and classifying alloys into their stable phases.

Dimensionality reduction can also be performed by the prediction data model unit 222. Dimensionality reduction simplifies complex data sets that contain high-dimensional data. Having too many features can make it more challenging for the model to learn effectively, as some features may be redundant or irrelevant. By reducing the number of features, the model focuses on the most important information, making it more efficient, thereby improving its accuracy. This process is especially useful when the number of features is larger than the amount of data, which can lead to overfitting. Simplifying the data helps the model generalize new data more effectively.

Optimization data model unit 224 can be used to perform multi-objective optimization where the objectives (as shown in FIG. 6) are often negatively correlated or in conflict. In such cases, multiple optimal solutions are possible. Genetic algorithms can be used to solve such problems, as they can handle conflicting goals efficiently. The effectiveness of such algorithms, however, is dependent on the accuracy of the machine learning models they rely on.

In some embodiments, sequential optimization techniques can be used to identify new alloys. Sequential optimization techniques used for the identification of new alloys can include, but are not limited to, Bayesian optimization and reinforcement learning. Sequential optimization focuses on identifying new alloys that the machine learning model predicts will have improved objective values (i.e., with respect to the particular application being optimized for). Predicted alloys can then be tested through simulations such as a multiscale simulation process described herein, and subsequently validated using experimental evaluation, such as experimental evaluation methods described herein.

The results of the simulation and validation can be inputted into the machine learning model as training data. The iterative process continuously refines the model, improving its accuracy and guiding the optimization to discover alloys with superior performance.

The simulation models unit 226 can be used to perform a multiscale simulation process. The multiscale simulation process can comprise simulation models based on traditional materials calculators. For example, in some embodiments, the simulation models can be configured as a computational quantum model to perform density functional theory (DFT) analysis. This DFT calculator can investigate the electronic structure of materials. DFT simplifies the calculation of the quantum mechanical states of a material. The simulation method can calculate various properties of a material system with reasonable computational efficiency such as a candidate alloy's initial stability and mechanical properties. Optionally, or in addition, the simulation model can be configured to simulate Ab-initio Molecular Dynamics (AIMD). This simulation method applies the DFT calculations at different finite ranges of temperatures.

In some embodiments, the simulation models can also be configured to perform a calculation of phase diagrams (i.e., CALPHAD). The simulation models can be used to model and predict thermodynamic properties and phase behavior of multi-component material systems. Different phases contribute to a variety of structural and functional properties of metallic systems. These different phases can be critical for determining the performance of an alloy in the real world. CALPHAD methods can also be configured to predict the melting behavior of different chemistries.

In some embodiments, the simulation models can be configured as phase field models. Phase field models can use computational techniques to simulate and predict the evolution of microstructures in materials. Phase field models can capture the complex interactions and transitions between different phases within a material by representing the microstructure as a continuous field that evolves over time. This approach can be particularly useful in developing new materials as it can help predict how different processing conditions and compositions will influence the resulting microstructure. By simulating the microstructural evolution, the phase field model can guide the design of materials with tailored properties.

In some embodiments, the simulation models can be configured to perform finite element modelling (i.e., FEM). Finite element modelling can be used to analyze and predict the behavior of materials and structures under various conditions. By breaking down a complex geometry into smaller, manageable elements, finite element modelling enables the detailed study of stress, strain, thermal properties, and other physical phenomena at a localized level. Finite element modelling can also be applied to various engineering problems, from mechanical stress analysis to heat transfer and fluid dynamics. Finite element modelling can simulate how materials will respond to different loading conditions, environmental factors, and design modifications. This is crucial for predicting material performance, optimizing design processes, and ensuring reliability before producing physical prototypes.

The validation unit 228 receives experimental evaluation data such as experimental data generated in a laboratory. The validation unit can provide recommendations to the optimization data model unit 224 to further refine the algorithm for determining alloy compositions.

In some embodiments, experimental evaluation can comprise an initial characterization of the alloy. This can involve testing lab-scale samples of the new alloy. Typically, arc-melting or casting is used to produce small alloy samples which are then cut and polished to expose the internal structure. The microstructure can be analyzed using optical microscopy and Scanning Electron Microscopy (SEM). One or more heat treatment (e.g., annealing) steps can be used to achieve the desired phases and microstructure. The sample can be cut again, polished, and re-examined through microscopy. In some cases, X-ray diffraction can be used to confirm the phases present and Electron Backscatter Diffraction (EBSD) can be conducted to verify the chemical composition. Optionally, or in addition, simple mechanical tests such as microhardness testing are performed to assess the material's mechanical properties.

In some embodiments, experimental evaluation can comprise standard qualification. Once the material's initial performance is validated, standardized testing can be used to establish a reliable benchmark for comparison. Regulatory bodies such as the American Society for Testing & Materials (ASTM), provide detailed protocols to assess various material properties. These procedures are designed to ensure that testing is consistent and repeatable, allowing for accurate comparisons across different materials. Following these standardized guidelines can be critical for industries, as it minimizes variability in test results and ensures that material performance can be reliably measured against established criteria. This uniformity also allows manufacturers to demonstrate compliance with industry standards, facilitating the material's adoption in broader applications.

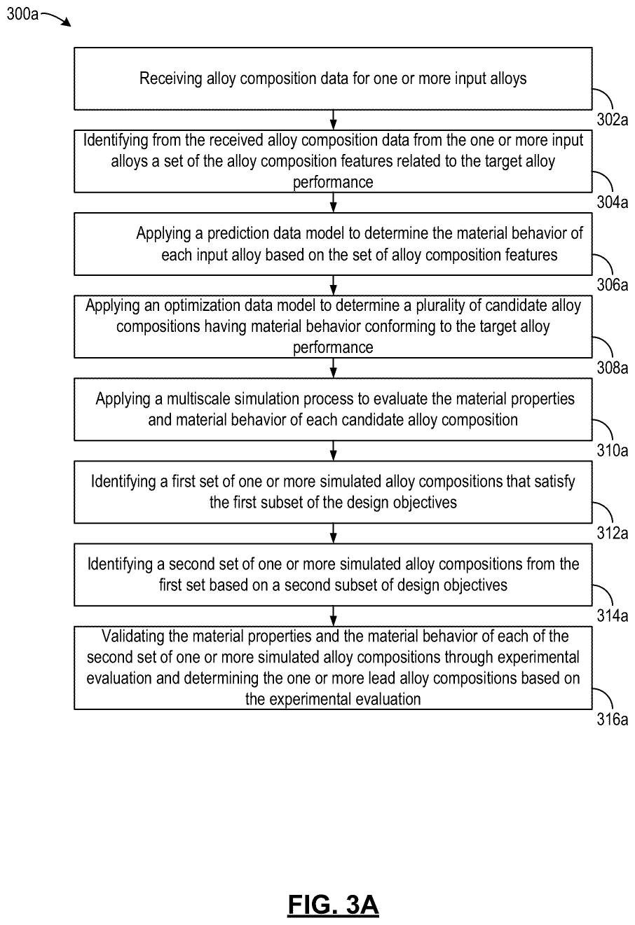

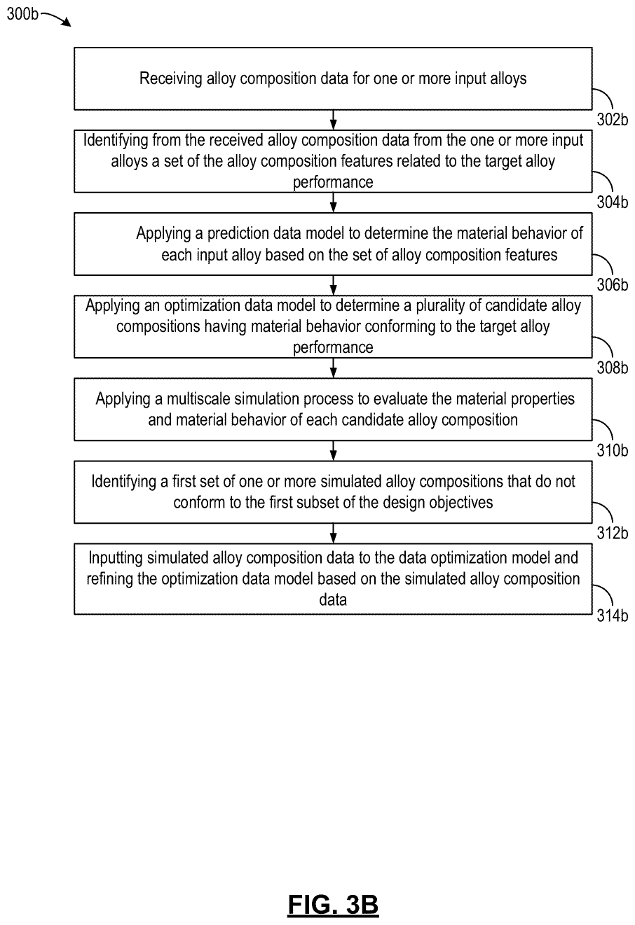

Reference is next made to FIG. 3A, shows a flow diagram of an example method 300a for alloy development in accordance with some embodiments. Method 300a describes a robust process for rapidly developing one or more alloys for a particular application based on a set of design requirements. Additionally, method 300a describes an iterative process that continues to be refined until one or more lead alloys can be determined.

The method begins at 302a with receiving alloy composition data from one or more input alloys. The system 100a can receive alloy composition data for one or more input alloys. Alloy composition data can include data corresponding to the material behavior of an alloy such as, for example, how a given alloy responds in various environments (e.g., how an alloy responds under high temperature, high pressure, or high alkalinity, etc.).

In some embodiments, the alloy composition data comprises simulated alloy composition data generated from a multiscale simulation process described herein. Simulated alloy composition data may be organized similarly to traditional alloy composition data (i.e., received from the experimental data). For example, simulated alloy composition data can include material properties and material behaviors for a given alloy.

The method proceeds to 304a with identifying from the received alloy composition data from the one or more input alloys a set of alloy composition features. The alloy composition features can be related to the target alloy performance. Alloy composition features correspond to material properties or material behaviors that are related to the target alloy performance. For example, material properties such as hardness and ductility may be important features that must be optimized for the particular application the alloy will be used in. Thus, alloy composition data can be cleaned, filtered, or pre-processed to extract these properties from the alloy composition data. Conversely, material properties such as thermal conductivity and electrical resistivity for example may not be important properties that need to be optimized. In such cases, such information may be omitted to ensure more accurate results.

The method proceeds to 306a with applying a prediction data model to determine the material behavior of each input alloy based on the set of alloy composition features. The prediction data model can uncover underlying relationships between material behavior based on the material properties of the alloy. For example, material properties in the alloy composition data that indicate that the alloy has high ductility can suggest that the alloy can undergo significant plastic deformation before failure.

The method proceeds to 308a with applying an optimization data model to determine a plurality of candidate alloy compositions. The plurality of candidate alloy compositions have material behavior conforming to the defined target alloy performance.

The method proceeds to 310a with applying a multiscale simulation process to evaluate the material properties and material behavior of each candidate alloy composition.

In some embodiments, applying a multiscale simulation process further comprises using generative machine learning-based structures as input. The machine learning-based structures may be based on the simulated alloy composition data from a multiscale simulation process. For example, the machine learning-based structures may be based on a previous iteration of the multiscale simulation in which the method identified a set of alloy compositions that did not conform to the first subset of design objectives.

The multiscale simulation process comprises at least two simulation models operating in a pre-defined sequence. In some embodiments, the at least two simulation models are based on machine learning methods. For example, the simulation models may be machine learning-based interatomic potentials models which can perform DFT calculations. As another example, the simulation models can be machine learning based phase field calculators which can replace or be used in combination with traditional phase field calculators.

In some embodiments, applying a multiscale simulation process further comprises sequencing the simulation models based on a material scale. As discussed, material behavior and material properties of alloys can be simulated on different scales. For example, an alloy can be analyzed at the atomic scale. At the atomic scale, the interactions between atoms such as bonding and crystal lattice structure (e.g., face-centered cubic, body centered-cubic, and or hexagonal close-packed) can determine properties such as strength and conductivity. As another example, alloys can be simulated at the nano scale (i.e., approximately between 1 to 100 nanometers in scale). As another example, alloys can be simulated at the micro scale (i.e., approximately between 1 micrometer to 1 millimeter in scale). As another example, alloys can be simulated at the mesoscale (i.e., approximately within the millimeter range). As another example, alloys can be simulated at the macro scale (i.e., greater than 1 mm). As another example, alloys can be simulated at the structural scale (i.e., the component or system level). It will be appreciated that alloys can be simulated at more material scales than the ones listed herein.

In some embodiments, the multiscale simulation process can sequence the simulation models based on an increasing material scale. For example, a first simulation model can be used to simulate an alloy at an atomic scale. Next, a second simulation model can be used to simulate the alloy at a nano scale. Additional simulation models can be used to simulate the alloy at other material scales as described herein (e.g., micro scale, mesoscale, macro scale). The pre-determined sequence may be defined to order the simulation models in increasing size of material scale. The pre-determined sequence may be defined to simulate all material scales or only a set of material scales depending on the application.

In some embodiments, the at least two simulation models can comprise at least four simulation models applied in a pre-determined sequence from an atomic scale to a macro scale. For example, the pre-determined sequence may comprise a first simulation model to simulate the material behavior of the alloy at the nano scale; a second simulation model to simulate the material behavior of the alloy at the micro scale; a third simulation model to simulate the material behavior of the alloy at the mesoscale; and a fourth simulation model to simulate the material behavior of the alloy at the macro scale.

The input of a second model is based on an output of a first simulation model in the pre-determined sequence. For example, material behavior that is determined from the first simulation model may be used as an input for the second simulation model. The first simulation model may be simulating the alloy at the nano scale. The second simulation model may be simulating the alloy at the micro scale. In order for the second simulation model to accurately simulate the alloy at the micro scale the second simulation model requires input from the first simulation model (i.e., material behavior at the nano scale). In this way, the relationship between scales can be understood to generate a more accurate representation of the material behavior of the alloy.

In some embodiments, an input of a third simulation model is based on the output from the first simulation model. For example, in cases where there are more than two simulation models in the pre-determined sequence, a third simulation model may require an output from the first simulation model. In this way, the first simulation model is not restricted to providing material behavior to only the second simulation model. The first simulation model can provide material behavior to any simulation model in the pre-determined sequence depending on the configuration of the multiscale simulation.

In some embodiments, the simulated alloy composition data generated from the multiscale simulation process is stored in the memory. The stored simulated alloy composition data can be used for subsequent iterations of the method 300a, as described herein.

The method proceeds to 312a with identifying a first set of one or more simulated alloy compositions that satisfy the first subset of the design objectives. In some embodiments, the first subset of design objectives corresponds to design objectives that can be validated through simulation. For example, the first subset of design objectives can correspond to material behaviors that can be validated using the multiscale simulation process described herein.

The method proceeds to 314a with identifying a second set of one or more simulated alloy compositions from the first set based on a second subset of design objectives.

In some embodiments, the second subset of design objectives corresponds to material behaviors associated with a feasibility of experimental evaluation such as factors that could limit the practical application of the alloy and/or hinder widespread adoption. For example, the second subset of design objectives may be related to the supply of a particular metallic component required for creating the alloy composition in the real world. In some cases, even if an alloy satisfies the first subset of design objectives, the alloy may not be feasible for experimental evaluation because it is too difficult to obtain (e.g., too expensive, difficult to procure). For example, alloys that are too difficult to produce in the real world or require numerous post-processing steps (such as heat treatments) or finishing before it can be utilized may not satisfy the second subset of design objectives.

The method proceeds to 316a with validating the material properties and the material behavior of each of the second set of one or more simulated alloy compositions through experimental evaluation. One or more lead alloy compositions can be determined based on the experimental evaluation.

In some embodiments, method steps 312a and 314a are not separate steps. Instead, after 310a, the method proceeds to identifying a set of one or more simulated alloy compositions that satisfy both the first subset and the second subset of design objectives. The method then proceeds to 316a, as discussed above. Reference is next made to FIG. 3B, shows a flow diagram of an example method 300b for alloy development in accordance with some embodiments. Method 300b describes a similar process to the method 300a described in FIG. 3A. Steps 302b, 304b, 306b, 308b, and 310b are identical to steps 302a, 304a, 306a, 308a, and 310a, respectively.

The method proceeds to 312b with identifying a first set of one or more simulated alloy compositions that do not conform to the first subset of the design objectives. The simulated alloy composition may not have any material properties or material behavior that are required for the particular application. In some cases, the simulated alloy composition may only have some material properties or material behavior that conforms to the first subset of design objectives. For example, the simulated alloy composition may have the desired hardness but may lack the desired ductility.

In some embodiments, the optimization data model can receive simulated alloy composition data as input. The simulated alloy composition data can be alloy composition generated through a multiscale simulation process described herein. In some cases, the simulated alloy composition data can correspond to alloy composition data from the set of identified alloy compositions that do not conform to the first subset of design objectives. For example, although these simulated alloy compositions will not be used to determine a lead alloy composition, the simulated alloy composition data may be useful input data for subsequent iterations of method 300a.

In some embodiments, the optimization data model can be refined based on the simulated alloy composition data. The simulated alloy composition data can be used as training data for the machine learning models. In this way, the multi-objective optimization model may be more accurate in a subsequent iteration.

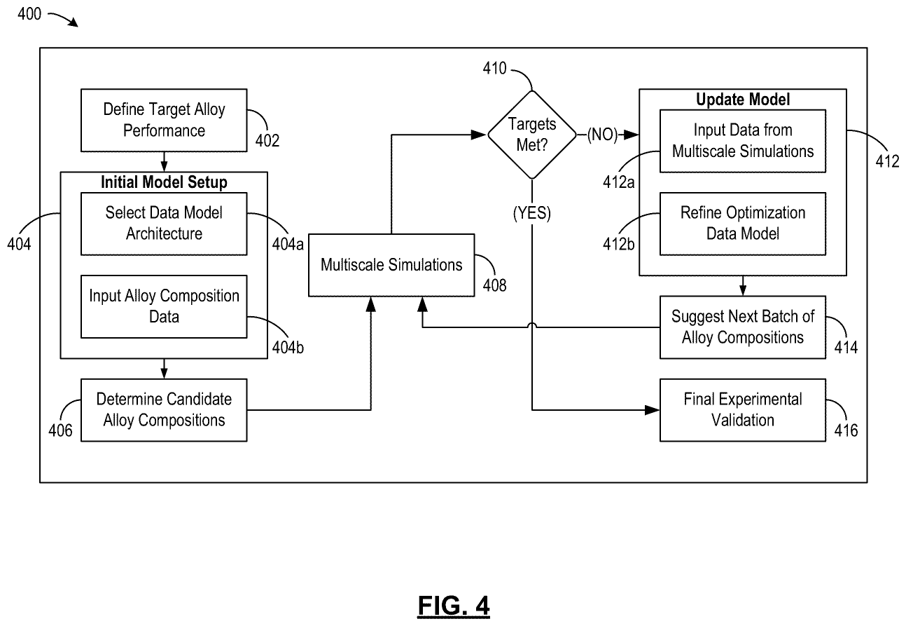

Reference is now made to FIG. 4, which shows a flow diagram for an alloy development process 400 in accordance with some embodiments. The flow diagram shows a representative implementation of the methods 300a and 300b described in FIGS. 3A and 3B, respectively. The process shown in FIG. 4 is described below using, as a non-limiting example, the development of a binder alloy for use in laser powder bed fusion (LPBF), with reference to the illustrative design objectives shown in FIG. 6.

The process begins at 402 with defining a target alloy performance. The target alloy performance defines the required material properties and/or material behaviors that make an alloy suitable for a particular process or application. Target alloy performance may include, for example, mechanical properties such as ductility, hardness, malleability, toughness, and cost of materials associated with particular process.

As a non-limiting example, alloy requirements can be defined by a set of design objectives such as those summarized in table 600 in FIG. 6, which illustrates desired target alloy performance criteria for an alloy suitable for use as a binder alloy in LPBF. It will be understood that the set of design objectives shown in FIG. 6 are provided for illustrative purposes only and that different applications may require different set of design objectives. For example, an alloy used for medical device applications may require a target alloy performance with a different set of design objectives.

The process continues at step 404 where the initial models are set up. At step 404a, the data model architecture is selected. Data models, such as a prediction data model and an optimization data model (or machine learning-based multi-objective optimization model), are selected and configured to develop one or more lead alloy compositions based on the target alloy performance defined in step 402. In the illustrated non-limiting example, the data model architecture is configured to develop one or more lead alloy compositions that could be suitable as a binder alloy in LPBF.

At step 404b, initial alloy composition data is provided as input for the data models. Alloy composition data can comprise experimental data available in the published literature as well as simulated alloy composition data (e.g., generated by the multiscale simulation process in a previous iteration of the alloy development process 400). In the illustrated non-limiting example, the input alloy composition data can correspond to alloys that have similar material properties and/or material behaviors as defined by a target alloy performance corresponding to a binder alloy that could be suitable in LPBF.

At step 406, configured data models determine a plurality of candidate alloy compositions that have material behavior conforming to the design objectives corresponding to the target alloy performance. In some embodiments, candidate alloy compositions conform to a first subset of design objectives of the set of design objectives. The first subset of design objectives may be, for example, design objectives that can be validated through simulation such as material properties and material behavior. In the illustrated, non-limiting example, the determined plurality of candidate alloy compositions may be suitable for additive manufacturing (e.g., 3D printing), exhibit sufficient wear resistance, and possess desired mechanical properties such as hardness and ductility.

At step 408, the selected initial alloy compositions are used in a multiscale simulation process.

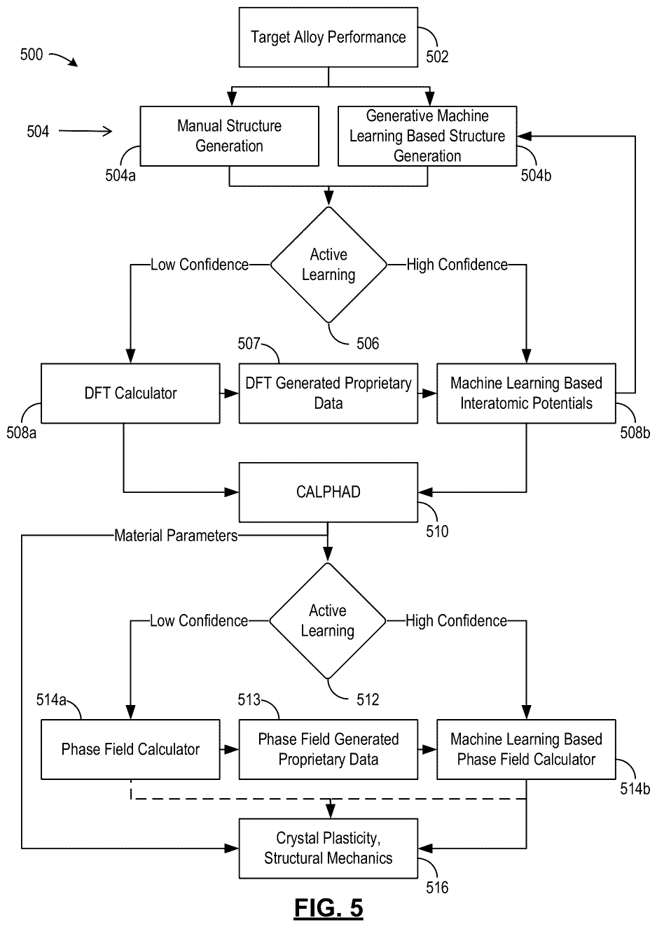

Reference is now made to FIG. 5, which shows a flow diagram 500 of an example multiscale simulation process in accordance with some embodiments. Generally, at each step, the multiscale simulation uses a simulation model that can be used for an increasing material scale. For example, at step 508a, DFT calculations are performed to compute electronic structure of materials at the atomistic scale. Later, at step 514a, phase field calculations are performed to measure structural information at the mesoscale.

With continued reference to the non-limiting example of FIG. 4, FIG. 5 illustrates a flow diagram 500 describing a multiscale simulation process for developing an example cemented carbide alloy. It will be understood that the cemented carbide alloy described herein is by way of example only, and that the multiscale simulation process can be applied to the development of various target alloy compositions having differing target alloy performances.

At step 502, performance criteria for a target alloy are defined. This performance criteria conforms to the target alloy performance defined at 402 by the alloy development process 400 of FIG. 4.

In the illustrated non-limiting example, the target alloy is a cemented carbide alloy with target criteria of enhanced wear resistance while maintaining good fracture toughness. The target alloy performance may further require that the alloy composition has good 3D printability (e.g., for applications such as involving additive manufacturing process). The target alloy may also be required to meet additional performance criteria as shown in Table 1. Table 1 also defines the expected ranges required for the target alloy to achieve the target alloy performance, as well as a column to describe the corresponding design criteria.

TABLE 1
Target alloy performance properties and their associated
target range for a cemented carbide alloy for
use in additive manufacturing processes

| Target alloy properties | Target range | Design criteria |
|---|---|---|
| Hardness | >8 HV | Wear resistance |
| Ductility parameter | >1 | Fracture toughness |
| Solidification interval | <100K | 3D printability |
| FCC_Ratio | >0.9 | Single phase solution |

At step 504, alloy structures are generated for target alloy compositions meeting the target performance criteria of step 502. Step 504 can include alloy structure generation based on a manual structure generation at step 504a. Optionally, and/or alternatively, alloy structure generation can be based on a generative machine learning-based structure at step 504b.

The manual structure generation at step 504a corresponds to alloy structures derived from alloy composition data available in experimental data. Generative machine learning-based structure generation at step 504b corresponds to simulated alloy composition data (e.g., generated by the multiscale simulation process in a previous iteration of the alloy development process 400).

Referring again to the illustrated non-limiting example of a cemented carbide alloy with performance criteria defined in Table 1, in one embodiment, a corresponding alloy structure is generated at step 504. This includes generating an atomistic cell unit that can satisfy the phase criteria (e.g., single phase solution) of a target cemented carbide alloy, as identified in step 502. For the target cemented carbide alloy, a face centered cubic (or FCC) structure may be desirable. Accordingly, at step 504, a FCC unit cell is generated with elements randomly assigned to each lattice position.

In some embodiments, the FCC unit cell is generated using the manual structure generation at step 504a. For example, a crystal graph generation model is used to generate a crystal graph based on the phase requirement.

In some other embodiments, the FCC unit cell is generated using one or more generative models in the ML-based structure generation at step 504b. For example, a trained machine learning-based interatomic potentials (MLIP) model is used at step 504b. In some examples, a generative model is queried to generate a candidate atomistic structure (e.g., a FCC structure).

In some embodiments, at step 504, the total energy of the candidate atomistic structure is also minimized. This process relaxes the atomic positions to reach a local minimum on the potential energy surface. A stable atomistic structure (e.g., based on energy minimization principles) can then be determined using a density functional theory (DFT) calculator at step 508a or an MLIP model at step 508b, as described herein.

Once the atomistic structure is generated at step 504, a determination is made at step 506 regarding the method to be used to compute the remaining properties. In various embodiments, determination is based on active learning algorithms. For example, the active learning algorithm can select the method to be used for computing energy and forces for a particular input structure, such as atomistic structure generated at step 504. The computation of energy and forces can be performed using either DFT analysis or a probabilistic machine learning-based interatomic potentials (MLIP) model. Unlike DFT, which provides a single energy value, a probabilistic MLIP model outputs a distribution of energy values, allowing for the computation of statistical quantities such as the mean and standard deviation.

In various embodiments, the active learning-based determination step of 506 leverages the statistical quantities provided by the MLIP model to determine whether to use DFT analysis or the MLIP model. For example, an active learning algorithm is used to evaluate the ratio of the mean to the standard deviation. If this ratio exceeds a predefined threshold, the MLIP model is used to compute the remaining properties at 508b; otherwise, DFT is selected to compute the remaining properties at 508a.

In various embodiments, descriptors/features can also be extracted from training data used for the MLIP model. Features can comprise distance between atoms (bond distance) and bond angles. Descriptors can comprise smooth overlap of atomic positions (SOAP). Nearest Neighbor methods can be used to calculate the distance between the new features and the training data features. If the distance is more than a specified and pre-determined threshold, then the DFT calculator is selected at 508a. Otherwise, the MLIP model is selected at 508b.

Referring to the illustrated non-limiting example, where after the atomistic structure of the cemented carbide alloy is generated at step 504, an active-learning based algorithm is used to compute the remaining properties. The generated atomistic structure is compared against the MLIP training set. If the atomistic structure is within a predefined tolerance, then the MLIP model is used to compute alloy properties at an atomistic scale at 508b. Otherwise, a DFT calculator is used to calculate the remaining properties at 508a.

In the context of the non-limiting example of a cemented carbide alloy, an example predefined tolerance threshold for determining whether the alloy is suitable for evaluation using an MLIP model can be a normalized mean atomic positional deviation in the range of 0.03 Å to 0.07 Å. In such an example, for the candidate atomistic structure, such as, the structure generated at step 504, the algorithm evaluates the three-dimensional atomic coordinates of all atoms in the structure. The algorithm then compares the atomic positions to the atomic positions of the structures contained within the MLIP training set. For each structure in the training set, the algorithm computes the positional deviation between corresponding atomic coordinates of the candidate structure and the training structure. A mean positional deviation is then calculated to quantify structural similarity. The resulting normalized mean positional deviation is compared against the predefined tolerance threshold (e.g., between 0.03 Å and 0.07 Å). If the computed mean deviation is less than or equal to the predefined tolerance threshold, then the candidate structure is deemed to be sufficiently similar to the training data, and the system elects to use the MLIP model. If the computed mean deviation exceeds the predefined tolerance threshold, then the structure is considered outside the reliable domain of the MLIP model, and a DFT calculator is instead selected. The tolerance threshold defines the operational boundary within which the MLIP model is considered reliable for evaluating cemented carbide atomistic structures.

If a DFT calculator is selected at step 508a, the DFT calculator determines an electronic structure of the material. The DFT workflow comprises determining an equation of states, relaxation, and determining properties.

In some embodiments, the output of the DFT calculator is stored as proprietary data at step 507. The DFT generated proprietary data can be used as training data for the MLIP model.

In the context of the non-limiting example of a cemented carbide alloy, the DFT calculator 508a, configured with a set of workflows that use complex quantum mechanics-based simulations, is used to compute the remaining properties of the alloy structure generated at 504. For example, the DFT calculator 508a can be used to compute hardness and ductility parameters for a given atomistic structure as generated at 504. For example, in one scenario, the DFT calculator 508a may output a hardness value of 7.8 and ductility parameter of 1.12. The properties determined by the DFT calculator 508a (e.g., the energy and forces) can then be stored in a proprietary database at step 507.

If a MLIP model is selected at 508b, the MLIP model performs molecular dynamics calculations for structural and mechanical properties of the alloy structure generated at 504. For example, using the proprietary data calculated for a cemented carbide alloy (e.g., generated by the DFT calculator in step 508a), a MLIP model can be trained to calculate target properties such as hardness and ductility.

Next, the process proceeds to step 510, where a simulation model is selected that models the thermodynamic properties of the alloy structure received from previous step 508a or 508b. For example, at step 510, a simulation model is selected to perform a calculation of phase diagrams (CALPHAD). For example, in the context of a cemented carbide alloy, key parameters such as free energies, computed solidification intervals, and FCC-phase ratios can be used as inputs to generate accurate phase diagrams.

The process then proceeds to step 512, where a simulation model for calculating phase field is selected. In various embodiments, the simulation model is selected based on an active learning algorithm, similar to the active learning algorithm described above at step 506. In addition to the active learning algorithm described at step 506, additional parameters are considered by the active learning algorithm at step 512 for determining whether to use a traditional phase field calculator at step 514a or a machine learning-based phase field calculator at step 514b. In some embodiments, the active learning algorithm considers material parameters (e.g., frozen range, partition coefficient, liquidous slope, diffusion coefficient) and/or manufacturing process information, etc. Manufacturing process information can include, for example, laser speed and power, solidification rate, and hatch distance, etc. Manufacturing process information can also be used to generate temperature fields for computational fluid dynamics.

In some embodiments, when the active learning algorithm determines that a traditional phase field calculator should be used at step 514a, the generated computational fluid dynamic information can be used by the traditional phase field calculator to generate microstructural information. In other embodiments, when the active learning algorithm determines that the machine learning-based phase field calculator should be used at step 514b, the generated computational fluid dynamic information can be used as parameters for the machine learning-based phase field calculator.

In some embodiments, nearest neighbor methods can be used to calculate the distance between input parameters for the configuration and the configurations in the training data for the machine learning-based phase field calculator. If the distance is above a specified and pre-determined threshold, then a phase field calculator is selected at step 514a. Otherwise, a machine learning-based phase field calculator is selected at step 514b.

In the context of the non-limiting example of a cemented carbide alloy, CALPHAD-derived alloy parameters are compared against the phase field machine learning training data to determine similarity. In one example implementation, each alloy is represented by N normalized parameters. The training data contains, for example, 3,000 cemented carbide alloys, forming a parameter matrix of size 3000×N. For a new cemented carbide alloy, CALPHAD is used to compute the same N normalized parameters. A mean difference is calculated between the parameter vector of the new alloy and the parameter matrix in the training data. The calculated mean deviation can be used by the active learning algorithm to determine whether the machine learning-based phase field calculator can be reliably applied.

If the traditional phase field calculator is selected at step 514a, a microscale simulation is conducted to assess how different cooling rates can affect the solidification process. For the non-limiting example of a cemented carbide, where additive manufacturing is the target process, performance of alloys made out of additive manufacturing techniques are highly dependent on the underlying process parameters. Phase field calculations at step 514a help assess how the process parameters can affect the microstructure of the cemented carbide material. For example, the phase field calculations at step 514a can determine how much of the desired FCC phase can be formed based on certain process parameters. Similarly, the phase field calculations at step 514a can determine how much time it will take for the microstructure to solidify based on material composition and process parameters.

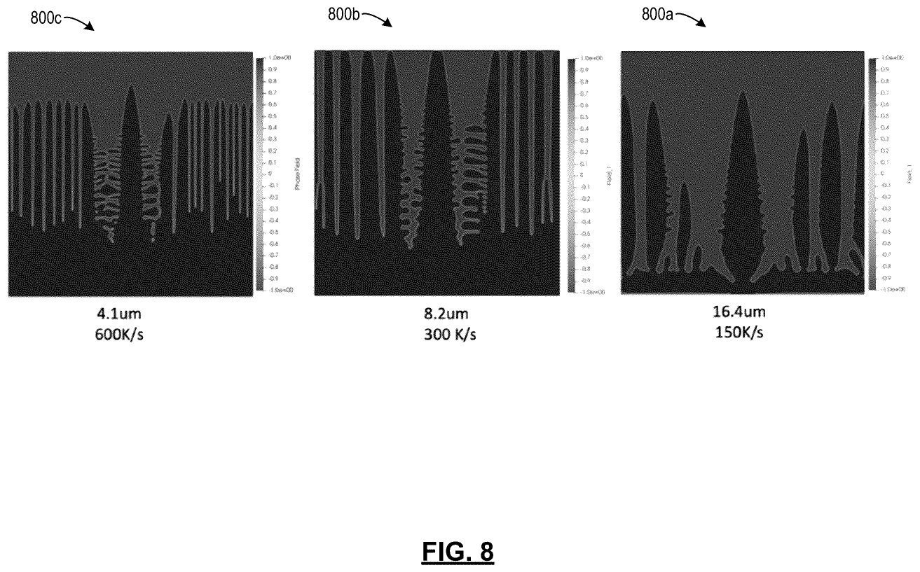

Reference is now made to FIG. 8, which shows example phase diagrams for an example alloy composition. The phase diagram can be an output of the phase field calculator used at 514a. In the non-limiting example shown in FIG. 8, the phase field calculator quantifies the relationship between cooling rate and resulting microstructural features. Desired microstructural features, such as for example, the effect of cooling rate on one of the microstructure features known as primary dendrite arm spacing (PDAS) can be extracted from the phase field diagrams shown in FIG. 8. As shown in FIG. 8 at a cooling rate of 150 K/s, the predicted PDAS is approximately 16.4 μm, as shown by phase diagram 800a; at a cooling rate of 300 K/s, the predicted PDAS decreases to approximately 8.2 μm, as shown by phase diagram 800b; and at a cooling rate of 600 K/s, the predicted PDAS further decreases to approximately 4.1 μm, as shown by phase diagram 800c. This shows the inverse relationship between cooling rate and PDAS captured by the phase field model.

Referring back to FIG. 5, in various embodiments, the output of the phase field calculator 514a is stored in a phase field generated database. The phase field generated data can be used as training data to train the machine learning based phase field calculator used at step 514b.

At step 513, a continuously evolving database can be created which can, for example, connect different alloys and process related parameters to their respective phase field trajectory (i.e., a series of images showing how an alloy is solidified during the solidification interval).

At step 514b, a machine learning based phase field calculator is selected to calculate microstructural parameters. Machine learning based calculators can be based on the proprietary data generated at step 513. Using a machine learning based phase field calculator can be advantageous as the computational acceleration aids in high throughput calculations.

The method proceeds to step 516, where microstructural information generated from the phase field calculator at step 514a or the machine learning-based phase field calculator at step 514b is used to generate microstructures for crystal plasticity (FEA) simulations. Crystal plasticity simulations are used to predict the mechanical properties of the alloy composition. In some embodiments, structural mechanics simulations are also performed at this stage based on the material parameters determined previously (e.g., specific material parameters determined at step 508a using the DFT calculator and/or at step 510 using the CALPHAD) to assess the final mechanical properties of the target alloy at a macro scale. Additionally, and/or optionally, the phase field trajectory as generated at step 514a and/or step 514b can be used as inputs to the structural simulation model. For the cemented carbide alloy example, stress-strain curves can be generated along with additional properties such as residual stresses, and hardness to assess whether a particular cemented carbide alloy composition would remain stable during the printing process (i.e., additive manufacturing process).

Referring back to FIG. 4, after the multiscale simulation process is completed at step 408, the method proceeds to step 410. At step 410, the system determines whether the target alloy performance is satisfied. If the target alloy performance is not satisfied, the process proceeds to step 412. At step 412a, the data models are updated with simulated alloy composition data generated from the multiscale simulations in step 408. At step 412b the simulated alloy composition data can be used as training data to refine multi-objective optimization model.

The method then proceeds to step 414, where the multi-objective optimization model generates a next set of candidate alloy compositions to be validated in the multiscale simulation process.

If the target alloy performance is satisfied at step 410, the method proceeds to step 416. At step 416, a final experimental evaluation is conducted to validate the properties of alloy compositions in a real-world setting.

As an example, experimental evaluation can comprise one or more of the following steps. The process may begin with procurement of required metallic components. In some embodiments, the required metallic components are procured in a powder form. Next, the element components are combined in their respective fractions. In some embodiments, the element components are combined through casting to produce casting rods. Next, the process includes evaluation of the microstructure of the casted rods. In some embodiments, the process includes heat treatment of the casted roads at specified temperatures. In some embodiments, the casted roads are heat treated for at least one hour for sample homogenization. The process next includes evaluation of the microstructure of the casted rods after heat treatment. The process proceeds to composition analysis to confirm the composition of the casted roads. In some embodiments, the composition analysis is conducted through XRD (X-ray Diffraction) and EDS (Energy-Dispersive X-ray Spectroscopy). In some embodiments, the process next proceeds to conduct a particle analysis to ensure good quality, size, and flowability. Next, the structural integrity of the alloy is confirmed through further analysis. In some embodiments, the structural integrity is configured through microstructural analysis (using, e.g., a scanning electron microscope (SEM)), density analysis (using, e.g., Archimedes test), and other mechanical property tests.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1. A method for developing one or more lead alloy compositions based on a set of design objectives defining a target alloy performance, the method comprising:
receiving, at a processor coupled to a memory, alloy composition data for one or more input alloys, the alloy composition data corresponding to a material behavior of each alloy and the one or more input alloys comprising material properties associated with a first subset of the design objectives;
identifying, at the processor, from the received alloy composition data from the one or more input alloys a set of the alloy composition features related to the target alloy performance;
applying, at the processor, a prediction data model to determine the material behavior of each input alloy based on the set of alloy composition features;
applying, at the processor, an optimization data model to determine a plurality of candidate alloy compositions having material behavior conforming to the first subset of the design objectives;
applying, at the processor, a multiscale simulation process to evaluate the material properties and material behavior of each candidate alloy composition, the multiscale simulation process comprising at least two simulation models operating in a pre-determined sequence, wherein an input of a second simulation model is based on an output of a first simulation model in the pre-determined sequence;
identifying, at the processor, a first set of one or more simulated alloy compositions that satisfy the first subset of the design objectives;
identifying, at the processor, a second set of one or more simulated alloy compositions from the first set based on a second subset of the design objectives; and
validating, at the processor, the material properties and the material behavior of each of the second set of one or more simulated alloy compositions through experimental evaluation and determining the one or more lead alloy compositions based on the experimental evaluation.

2. The method of claim 1, wherein alloy composition data comprises simulated alloy composition data generated from the multiscale simulation process.

3. The method of claim 1, wherein applying, at the processor, the optimization data model further comprises:
identifying a set of alloy compositions that do not conform to the first subset of the design objectives; and
inputting simulated alloy composition data to the data optimization model; and
refining the optimization data model based on the simulated alloy composition data.

4. The method of claim 1, wherein the optimization data model is configured to assess at least two of the material properties in the first subset of the design objectives.

5. The method of claim 1, wherein applying the multiscale simulation process, at the processor, further comprises inputting generative machine learning-based structures based on the simulated alloy composition data.

6. The method of claim 1, wherein applying the multiscale simulation process, at the processor, further comprises sequencing the at least two simulation models based on a material scale.

7. The method of claim 1, wherein the at least two simulation models further comprise at least four simulation models applied in a pre-determined sequence from an atomic-scale to a macro-scale.

8. The method of claim 1, wherein at least one of the at least two simulation models is based on machine learning methods.

9. The method of claim 1, wherein applying, at the processor, the multiscale simulation process further comprises selecting the simulation models for the pre-determined sequence based on accuracy and speed.

10. The method of claim 1, wherein applying, at the processor, the multiscale simulation process further comprises selecting the simulation models for the pre-determined sequence based on a scale of atoms.

11. The method of claim 1, wherein an input of a third simulation model is based on the output from the first simulation model.

12. The method of claim 1, wherein the simulated alloy composition data generated from the multiscale simulation process is stored in the memory.

13. The method of claim 1, wherein the second subset of the design objectives corresponds to material behaviors associated with feasibility of experimental evaluation.

14. A system for developing one or more lead alloy compositions based on a set of design objectives defining a target alloy performance, the system comprising:
a prediction data model;
an optimization data model;
at least two simulation models; and
a processor coupled to a memory, the processor configured to:
receive alloy composition data for one or more input alloys, the alloy composition data corresponding to a material behavior of each alloy and the one or more input alloys comprising material properties associated with a first subset of the design objectives;
identify from the received alloy composition data from the one or more input alloys a set of the alloy composition features related to the target alloy performance;
apply the prediction data model to determine the material behavior of each input alloy based on the set of alloy composition features;
apply an optimization data model to determine a plurality of candidate alloy compositions having material behavior conforming to the first subset of the design objectives;
apply a multiscale simulation process to evaluate the material properties and material behavior of each candidate alloy composition, the multiscale simulation process comprising at least two simulation models operating in a pre-determined sequence, wherein an input of a second simulation model is based on an output of a first simulation model in the pre-determined sequence;
identify a first set of one or more simulated alloy compositions that satisfy the first subset of the design objectives;
identify a second set of one or more simulated alloy compositions from the first set based on a second subset of the design objectives; and
receive experimental validation data comprising material behavior of each of the second set of one or more simulated alloy compositions and provide recommendations for determining one or more lead alloy compositions based on the experimental validation data.

15. The system of claim 14, wherein alloy composition data comprises simulated alloy composition data generated from the multiscale simulation process.

16. The system of claim 14, wherein the processor is further configured to apply the optimization data model to:
identify a set of alloy compositions that do not conform to the first subset of the design objectives; and
input simulated alloy composition data to the data optimization model; and
refine the optimization data model based on the simulated alloy composition data.

17. The system of claim 14, wherein the processor is further configured to apply the optimization data model to assess at least two of the material properties in the first subset of the design objectives.

18. The system of claim 14, wherein the processor is further configured to apply the multiscale simulation process to input generative machine learning-based structures based on the simulated alloy composition data.

19. The system of claim 14, wherein the processor is further configured to apply the multiscale simulation process to sequence the simulation models based on a material scale.

20. The system of claim 14, wherein the at least two simulation models further comprise at least four simulation models applied in a pre-determined sequence from an atomic-scale to a macro-scale.

21. The system of claim 14, wherein at least one of the at least two simulation models is based on machine learning methods.

22. The system of claim 14, wherein the processor is further configured to apply the multiscale simulation process to select the simulation models for the pre-determined sequence based on accuracy and speed.

23. The system of claim 14, wherein the processor is further configured to apply the multiscale simulation process to select the simulation models for the pre-determined sequence based on a scale of atoms.

24. The system of claim 14, wherein an input of a third simulation model is based on the output from the first simulation model.

25. The system of claim 14, wherein the simulated alloy composition data generated from the multiscale simulation process is stored in the memory.

26. The system of claim 14, wherein the second subset of the design objectives corresponds to material behaviors associated with a feasibility of experimental evaluation.
